SEQUENCE LISTING (7 sequences)

SEQ ID NO: 1 - protein, 574 aa, Artificial Sequence
  RSV F protein A2 full length sequence
MELLILKANAITTILTAVTFCFASGQNITEEFYQSTCSAVSKGYLSALRTGWYTSVITIELSNIKKNKCNGTDAKIKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 2 - protein, 27 aa, Artificial Sequence
  Trimerization domain
GYIPEAPRDGQAYVRKDGEWVLLSTFL

SEQ ID NO: 3 - protein, 4 aa, Artificial Sequence
  Linker
SAIG

SEQ ID NO: 4 - protein, 574 aa, Artificial Sequence
  RSV preF2.1
MELLILKANAITTILTAVTFCFASGQNITEEFYQSTCSAVSKGYLGALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 5 - protein, 574 aa, Artificial Sequence
  RSV preF2.2
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 6 - DNA, 1725 bp, Artificial Sequence
  RSV F pre-F2.1
atggagctgctgatcctgaaggccaacgccatcaccaccatcctgaccgccgtgaccttctgcttcgccagcggccagaacatcaccgaggagttctaccagagcacctgcagcgccgtgagcaagggctacctgggcgccctgagaaccggctggtacaccagcgtgatcaccatcgagctgagcaacatcaaggagatcaagtgcaacggcaccgacgccaaggtgaagctgatcaagcaggagctggacaagtacaagaacgccgtgaccgagctgcagctgctgatgcagagcacccccgccaccaacaacagagccagaagagagctgcccagattcatgaactacaccctgaacaacgccaagaagaccaacgtgaccctgagcaagaagagaaagagaagattcctgggcttcctgctgggcgtgggcagcgccatcgccagcggcgtggccgtgagcaaggtgctgcacctggagggcgaggtgaacaagatcaagagcgccctgctgagcaccaacaaggccgtggtgagcctgagcaacggcgtgagcgtgctgaccagcaaggtgctggacctgaagaactacatcgacaagcagctgctgcccatcgtgaacaagcagagctgcagcatccccaacatcgagaccgtgatcgagttccagcagaagaacaacagactgctggagatcaccagagagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgagacagcagagctacagcatcatgagcatcatcaaggaggaggtgctggcctacgtggtgcagctgcccctgtacggcgtgatcgacaccccctgctggaagctgcacaccagccccctgtgcaccaccaacaccaaggagggcagcaacatctgcctgaccagaaccgacagaggctggtactgcgacaacgccggcagcgtgagcttcttcccccaggccgagacctgcaaggtgcagagcaacagagtgttctgcgacaccatgaacagcctgaccctgcccagcgaggtgaacctgtgcaacgtggacatcttcaaccccaagtacgactgcaagatcatgaccagcaagaccgacgtgagcagcagcgtgatcaccagcctgggcgccatcgtgagctgctacggcaagaccaagtgcaccgccagcaacaagaacagaggcatcatcaagaccttcagcaacggctgcgactacgtgagcaacaagggcgtggacaccgtgagcgtgggcaacaccctgtactacgtgaacaagcaggagggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgagctgctgcacaacgtgaacgccgtgaagagcaccaccaacatcatgatcaccaccatcatcatcgtgatcatcgtgatcctgctgagcctgatcgccgtgggcctgctgctgtactgcaaggccagaagcacccccgtgaccctgagcaaggaccagctgagcggcatcaacaacatcgccttcagcaactga

SEQ ID NO: 7 - DNA, 1725 bp, Artificial Sequence
  RSV F pre-F2.2
atggagctgctgatcctgaaggccaacgccatcaccaccatcctgaccgccgtgaccttctgcttcgccagcggccagaacatcaccgaggagttctaccagagcacctgcagcgccgtgagcaagggctacctgagcgccctgagaaccggctggtacaccagcgtgatcaccatcgagctgagcaacatcaaggagatcaagtgcaacggcaccgacgccaaggtgaagctgatcaagcaggagctggacaagtacaagaacgccgtgaccgagctgcagctgctgatgcagagcacccccgccaccaacaacagagccagaagagagctgcccagattcatgaactacaccctgaacaacgccaagaagaccaacgtgaccctgagcaagaagagaaagagaagattcctgggcttcctgctgggcgtgggcagcgccatcgccagcggcgtggccgtgagcaaggtgctgcacctggagggcgaggtgaacaagatcaagagcgccctgctgagcaccaacaaggccgtggtgagcctgagcaacggcgtgagcgtgctgaccagcaaggtgctggacctgaagaactacatcgacaagcagctgctgcccatcgtgaacaagcagagctgcagcatccccaacatcgagaccgtgatcgagttccagcagaagaacaacagactgctggagatcaccagagagttcagcgtgaacgccggcgtgaccacccccgtgagcacctacatgctgaccaacagcgagctgctgagcctgatcaacgacatgcccatcaccaacgaccagaagaagctgatgagcaacaacgtgcagatcgtgagacagcagagctacagcatcatgagcatcatcaaggaggaggtgctggcctacgtggtgcagctgcccctgtacggcgtgatcgacaccccctgctggaagctgcacaccagccccctgtgcaccaccaacaccaaggagggcagcaacatctgcctgaccagaaccgacagaggctggtactgcgacaacgccggcagcgtgagcttcttcccccaggccgagacctgcaaggtgcagagcaacagagtgttctgcgacaccatgaacagcctgaccctgcccagcgaggtgaacctgtgcaacgtggacatcttcaaccccaagtacgactgcaagatcatgaccagcaagaccgacgtgagcagcagcgtgatcaccagcctgggcgccatcgtgagctgctacggcaagaccaagtgcaccgccagcaacaagaacagaggcatcatcaagaccttcagcaacggctgcgactacgtgagcaacaagggcgtggacaccgtgagcgtgggcaacaccctgtactacgtgaacaagcaggagggcaagagcctgtacgtgaagggcgagcccatcatcaacttctacgaccccctggtgttccccagcaacgagttcgacgccagcatcagccaggtgaacgagaagatcaaccagagcctggccttcatcagaaagagcgacgagctgctgcacaacgtgaacgccgtgaagagcaccaccaacatcatgatcaccaccatcatcatcgtgatcatcgtgatcctgctgagcctgatcgccgtgggcctgctgctgtactgcaaggccagaagcacccccgtgaccctgagcaaggaccagctgagcggcatcaacaacatcgccttcagcaactga

(19) United States
(12) Patent Application Publication
(10) Pub. No.: US 2022/0273787 A1
(43) Pub. Date: Sep. 1, 2022

(54) CO-ADMINISTRATION OF SEASONAL INFLUENZA VACCINE AND AN ADENOVIRUS BASED RESPIRATORY SYNCYTIAL VIRUS VACCINE

(71) Applicant: Janssen Vaccines & Prevention B.V., Leiden (NL)

(72) Inventors: Benoit Christophe Stephan CALLENDRET, Den Haag (NL); Jerald C. SADOFF, Washington, DC (US); Els DE PAEPE, Lokeren (BE)

(21) Appl. No.: 17/595,255

(22) PCT Filed: May 14, 2020
(86) PCT No.: PCT/EP2020/063406

§ 371 Date: Nov. 12, 2021

Related U.S. Application Data
(60) Provisional application No. 62/848,036, filed on May 15, 2019.

(51) Int. Cl. A61K 39/12; A61P 31/14; A61P 31/16; C12N 7/00; C12N 15/86; A61K 39/145

(52) U.S. Cl. CPC A61K 39/12; A61P 31/14; A61P 31/16; C12N 7/00; C12N 15/86; A61K 39/145; A61K 2039/54

(57) ABSTRACT

Methods of inducing a protective immune response against respiratory syncytial virus (RSV) and against influenza virus, without inducing a severe adverse event in human subjects are described. The methods include administering to the subjects an effective amount of an adenoviral vector encoding a recombinant RSV F polypeptide that is stabilized in a pre-fusion conformation, along with an effective amount of an influenza vaccine.

### FIELD OF THE INVENTION

The present invention is in the field of medicine. In particular, embodiments of the invention relate to adenovirus-based vaccines and uses thereof in combination with influenza vaccine for prophylactic treatment of Respiratory Syncytial Virus (RSV) and influenza virus infections.

### BACKGROUND

Respiratory syncytial virus (RSV) is considered to be the most important cause of serious acute respiratory illness in infants and children under 5 years of age (Hall, et al., N Engl J Med. 2009:360;588-598; Shay et al., JAMA. 1999:282;1440-1446; Stockman et al., Pediatr Infect Dis J. 2012:31;5-9). Globally, RSV is responsible for an estimated 3.4 million hospitalizations annually. In the United States, RSV infection in children under 5 years of age is the cause of 57,000 to 175,000 hospitalizations, 500,000 emergency room visits, and approximately 500 deaths each year (Paramore et al., Pharmacoeconomics. 2004:22;275-284; Shay et al., JAMA. 1999:282;1440-1446; Stockman et al., Pediatr Infect Dis J. 2012:31;5-9). In the US, 60% of infants are infected upon initial exposure to RSV (Glezen et al., Am J Dis Child. 1986:140;543-546), and nearly all children will have been infected with the virus by 2-3 years of age. Immunity to RSV is transient, and repeated infection occurs throughout life (Hall et al., J Infect Dis. 1991:163;693-698). In children under 1 year of age, RSV is the most important cause of bronchiolitis, and RSV hospitalization is highest among children under 6 months of age (Centers for Disease Control and Prevention (CDC). Respiratory Syncytial Virus Infection (RSV)—Infection and Incidence. Available at: http://www.cdc.gov/rsv/about/infection.html (last accessed 2 Jun. 2016); Hall, et al., N Engl J Med. 2009:360;588-598). Almost all RSV-related deaths (99%) in children under 5 years of age occur in the developing world (Nair et al., Lancet. 2010:375;1545-1555). Nevertheless, the disease burden due to RSV in developed countries is substantial, with RSV infection during childhood linked to the development of wheezing, airway hyperreactivity and asthma (Peebles et al., J Allergy Clin Immunol. 2004:113; S15-18; Regnier and Huels, Pediatr Infect Dis J. 2013:32;820-826; Sigurs et al., Am J Respir Crit Care Med. 2005:171;137-141; Simoes et al., J Allergy Clin Immunol. 2010:126;256-262; Simoes et al., J Pediatr. 2007:151;34-42, 42 e31).

In addition to children, RSV is an important cause of respiratory infections in the elderly, immunocompromised, and those with underlying chronic cardio-pulmonary conditions (Falsey et al., N Engl J Med. 2005:352;1749-1759). In long-term care facilities, RSV is estimated to infect 5-10% of the residents per year with significant rates of pneumonia (10 to 20%) and death (2 to 5%) (Falsey et al., Clin Microbiol Rev. 2000:13;371-384). In one epidemiology study of RSV burden, it was estimated that 11,000 elderly persons die annually of RSV in the US (Thompson et al., JAMA. 2003:289;179-186). These data support the importance of developing an effective vaccine for certain adult populations.

Prophylaxis through passive immunization with a neutralizing monoclonal antibody against the RSV fusion (F) glycoprotein (Synagis® [palivizumab]) is available, but only indicated for premature infants (less than 29 weeks gestational age), children with severe cardio-pulmonary disease or those that are profoundly immunocompromised (American Academy of Pediatrics Committee on Infectious Diseases, American Academy of Pediatrics Bronchiolitis Guidelines Committee. Updated guidance for palivizumab prophylaxis among infants and young children at increased risk of hospitalization for respiratory syncytial virus infection. Pediatrics. 2014:134;415-420). Synagis has been shown to reduce the risk of hospitalization by 55% (Prevention. Prevention of respiratory syncytial virus infections: indications for the use of palivizumab and update on the use of RSV-IGIV. American Academy of Pediatrics Committee on Infectious Diseases and Committee of Fetus and Newborn. Pediatrics. 1998:102;1211-1216).

Despite the high disease burden and a strong interest in RSV vaccine development, no licensed vaccine is available for RSV. In the late 1960s, a series of studies were initiated to evaluate a formalin-inactivated RSV vaccine (FI-RSV) adjuvanted with alum, and the results of these studies had a major impact on the RSV vaccine field. Four studies were performed in parallel in children of different age groups with an FI-RSV vaccine delivered by intramuscular injection (Chin et al., Am J Epidemiol. 1969:89;449-463; Fulginiti et al., Am J Epidemiol. 1969:89;435-448; Kapikian et al., Am J Epidemiol. 1969:89;405-421; Kim et al., Am J Epidemiol. 1969:89;422-434). Eighty percent of the RSV-infected FI-RSV recipients required hospitalization and two children died during the next winter season (Chin et al., Am J Epidemiol. 1969:89;449-463). Only 5% of the children in the RSV-infected control group required hospitalization. The mechanisms of the observed enhanced respiratory disease (ERD) among the FI-RSV recipients upon reinfection have been investigated and are believed to be the result of an aberrant immune response in the context of small bronchi present in that age group. Data obtained from analysis of patient samples and animal models suggest that FI-RSV ERD is characterized by low neutralizing antibody titers, the presence of low avidity non-neutralizing antibodies promoting immune complex deposition in the airways, reduced cytotoxic CD8+ T-cell priming shown to be important for viral clearance, and enhanced CD4+ T helper type 2 (Th2)-skewed responses with evidence of eosinophilia (Beeler et al., Microb Pathog. 2013:55;9-15; Connors et al., J Virol. 1992:66;7444-7451; De Swart et al., J Virol. 2002:76;11561-11569; Graham et al., J Immunol. 1993:151;2032-2040; Kim et al., Pediatr Res. 1976:10;75-78; Murphy et al., J Clin Microbiol. 1986:24;197-202; Murphy et al., J Clin Microbiol. 1988:26;1595-1597; Polack et al., J Exp Med. 2002:196;859-865). It is believed that the chemical interaction of formalin and RSV protein antigens may be one of the mechanisms by which the FI-RSV vaccine promoted ERD upon subsequent RSV infection (Moghaddam et al., Nat Med. 2006:12;905-907). For these reasons, formalin is no longer used in RSV vaccine development.

In addition to the FI-RSV vaccine, several live-attenuated and subunit RSV vaccines have been examined in animal models and human studies, but many have been inhibited by the inability to achieve the right balance of safety and immunogenicity/efficacy. Live-attenuated vaccines have been specifically challenged by difficulties related to over- and under-attenuation in infants (Belshe et al., J Infect Dis. 2004:190;2096-2103; Karron et al., J Infect Dis. 2005:191;1093-1104; Luongo et al., Vaccine. 2009:27;5667-5676). With regard to subunit vaccines, the RSV fusion (F) and glycoprotein (G) proteins, which are both membrane proteins, are the only RSV proteins that induce neutralizing antibodies (Shay et al., JAMA. 1999:282;1440-1446). Unlike the RSV G protein, the F protein is conserved between RSV strains. A variety of RSV F-subunit vaccines have been developed based on the known superior immunogenicity, protective immunity and the high degree of conservation of the F protein between RSV strains (Graham, Immunol Rev. 2011:239;149-166). The proof-of-concept provided by the currently available anti-F protein neutralizing monoclonal antibody prophylaxis provides support for the idea that a vaccine inducing high levels of long-lasting neutralizing antibody may prevent RSV disease (Feltes et al., Pediatr Res. 2011:70;186-191; Groothuis et al., J Infect Dis. 1998:177;467-469; Groothuis et al., N Engl J Med. 1993:329;1524-1530). Several studies have suggested that decreased protection against RSV in elderly could be attributed to an age-related decline in interferon gamma (IFNγ) production by peripheral blood mononuclear cells (PBMCs), a reduced ratio of CD8+ to CD4+ T cells, and reduced numbers of circulating RSV-specific CD8+ memory T cells (De Bree et al., J Infect Dis. 2005:191;1710-1718; Lee et al., Mech Ageing Dev. 2005:126;1223-1229; Looney et al., J Infect Dis. 2002:185;682-685). High levels of serum neutralizing antibody are associated with less severe infections in elderly (Walsh and Falsey, J Infect Dis. 2004:190;373-378). It has also been demonstrated that, following RSV infection in adults, serum antibody titers rise rapidly but then slowly return to pre-infection levels after 16 to 20 months (Falsey et al., J Med Virol. 2006:78;1493-1497). With consideration given to the previously observed ERD in the FI-RSV vaccine studies in the 1960s, future vaccines should promote a strong antigen-specific CD8+ T-cell response and avoid a skewed Th2-type CD4+ T cell response (Graham, Immunol Rev. 2011:239;149-166).

RSV F protein fuses the viral and host-cell membranes by irreversible protein refolding from the labile pre-fusion conformation to the stable post-fusion conformation. Structures of both conformations have been determined for RSV F (McLellan et al., Science 2013:342, 592-598; McLellan et al., Nat Struct Mol Biol 2010:17, 248-250; McLellan et al., Science 340, 2013:1113-1117; Swanson et al., Proceedings of the National Academy of Sciences of the United States of America 2011:108, 9619-9624), as well as for the fusion proteins from related paramyxoviruses, providing insight into the mechanism of this complex fusion machine. Like other type I fusion proteins, the inactive precursor, RSV F0, requires cleavage during intracellular maturation by a furin-like protease. RSV F0 contains two furin sites (e.g., between amino acid residues 109/110 and 136/137 of the RSV F0 with a GenBank accession No. ACO83301), which leads to three polypeptides: F2, p27 and F1, with the latter containing a hydrophobic fusion peptide (FP) at its N-terminus. To refold from the pre-fusion to the post-fusion conformation, the refolding region 1 (RR1) (e.g., between residue 137 and 216, that includes the FP and heptad repeat A (HRA)) has to transform from an assembly of helices, loops and strands to a long continuous helix. The FP, located at the N-terminal segment of RR1, is then able to extend away from the viral membrane and insert into the proximal membrane of the target cell. Next, the refolding region 2 (RR2), which forms the C-terminal stem in the pre-fusion F spike and includes the heptad repeat B (HRB), relocates to the other side of the RSV F head and binds the HRA coiled-coil trimer with the HRB domain to form the six-helix bundle. The formation of the RR1 coiled-coil and relocation of RR2 to complete the six-helix bundle are the most dramatic structural changes that occur during the refolding process.

Most neutralizing antibodies in human sera are directed against the pre-fusion conformation, but due to its instability the pre-fusion conformation has a propensity to prematurely refold into the post-fusion conformation, both in solution and on the surface of the virions. RSV F polypeptides stabilized in a pre-fusion conformation are described. See, e.g., WO2014/174018, WO2014/202570 and WO 2017/174564. However, there is no report on the safety, efficacy/immunogenicity of such polypeptides in humans.

Influenza viruses are major human pathogens, causing a respiratory disease (commonly referred to as “influenza” or “the flu”) that ranges in severity from sub-clinical infection to primary viral pneumonia which can result in death. The clinical effects of infection vary with the virulence of the influenza strain and the exposure, history, age, and immune status of the host. Every year it is estimated that approximately 1 billion people worldwide undergo infection with influenza virus, leading to severe illness in 3-5 million cases and an estimated 300,000 to 500,000 of influenza related deaths.

There are three genera of influenza virus (types A, B and C) responsible for infectious pathologies in humans and animals. The type A and type B viruses are the agents responsible for the influenza seasonal epidemics (type A and B) and pandemics (type A) observed in humans.

Influenza A viruses can be classified into influenza virus subtypes based on variations in antigenic regions of two genes that encode the surface glycoproteins hemagglutinin (HA) and neuraminidase (NA) which are required for viral attachment and cellular release, respectively. Currently, sixteen subtypes of HA (H1-H16) and nine NA (N1-N9) antigenic variants are known in influenza A virus. Only some of the influenza A subtypes (i.e. H1N1, H1N2 and H3N2) circulate among people, but all combinations of the 16 HA and 9 NA subtypes have been identified in animals, in particular, in avian species.

The influenza type B virus strains are strictly human. The antigenic variation in HA within the influenza type B virus strains is smaller than those observed within the type A strains. Two genetically and antigenically distinct lineages of influenza B virus are circulating in humans, as represented by the B/Yamagata/16/88 (also referred to as B/Yamagata) and B/Victoria/2/87 (B/Victoria) lineages (Ferguson et al., Nature. 2003 Mar 27;422(6930):428-33.). Although the spectrum of disease caused by influenza B viruses is generally milder than that caused by influenza A viruses, severe illness requiring hospitalization is still frequently observed with influenza B infection.

Due to the highly variable and mutable nature of influenza antigens, developing a vaccine has proven difficult. However, vaccination is the most proven method for protecting against the disease and its serious complications. The vaccine must be reformulated and re-administered each year in anticipation of the serotypes of the virus predicted to be prevalent in a population each flu season and is therefore considered a “seasonal” vaccine. Typically, the most common human vaccine is a combination of one representative strain from each of the principal viral types predominantly responsible for annual global influenza outbreaks since 1977, including A (H1N1), A (H3N2) and B. There are two classes of influenza vaccine, including trivalent inactivated vaccine (TIV), given by intramuscular (IM) injection to individuals aged 6 months and older, and live attenuated influenza virus vaccine (LAIV), administered intranasally in healthy, non-pregnant persons aged 2-49.

A population susceptible to RSV infection is often also susceptible to influenza virus infection. Thus, there is a need for a safe and effective method for coadministration of a vaccine against RSV and a vaccine for influenza virus in a subject in need thereof.

### SUMMARY OF THE INVENTION

In one general aspect, the present application describes a method for inducing both a protective immune response against respiratory syncytial virus (RSV) infection and a protective immune response against influenza virus infection in a human subject in need thereof, comprising intramuscularly administering to the subject (a) an effective amount of a pharmaceutical composition, preferably a vaccine, comprising an adenoviral vector comprising a nucleic acid encoding an RSV F polypeptide that is stabilized in a pre-fusion conformation, wherein the effective amount of the pharmaceutical composition comprises about 1×10¹⁰ to about 1×10¹² viral particles of the adenoviral vector per dose, and (b) an effective amount of an influenza vaccine, wherein (a) and (b) are co-administered.

In certain embodiments, the pharmaceutical composition of (a) and the vaccine of (b) are administered at the same time.

In certain embodiments, the adenoviral vector is replication-incompetent and has a deletion in at least one of the adenoviral early region 1 (E1 region) and the early region 3 (E3 region).

In certain embodiments, the adenoviral vector is a replication-incompetent Ad26 adenoviral vector having a deletion of the E1 region and the E3 region.

In certain embodiments, the adenoviral vector is a replication-incompetent Ad35 adenoviral vector having a deletion of the E1 region and the E3 region.

In certain embodiments, the recombinant RSV F polypeptide encoded by the adenoviral vector has the amino acid sequence of SEQ ID NO: 4 or SEQ ID NO: 5.

In certain embodiments, the nucleic acid encoding the RSV F polypeptide comprises the polynucleotide sequence of SEQ ID NO: 6 or SEQ ID NO: 7.

In certain embodiments, the effective amount of the pharmaceutical composition comprises about 1×10¹¹ viral particles of the adenoviral vector per dose.

In certain embodiments, the influenza vaccine is a seasonal influenza vaccine.

In certain embodiments, the subject is susceptible to the RSV infection.

In certain embodiments, the subject is susceptible to the influenza virus infection.

In certain embodiments, the protective immune response is characterized by an absent or reduced RSV clinical symptom in the subject upon exposure to RSV.

In certain embodiments, the protective immune response is characterized by an absent or reduced influenza virus clinical symptom in the subject upon exposure to influenza virus.

In certain embodiments, the protective immune response is characterized by neutralizing antibodies to RSV and/or protective immunity against RSV.

In certain embodiments, the protective immune response is characterized by neutralizing antibodies to influenza virus and/or protective immunity against influenza virus.

In certain embodiments, the administration does not induce any severe adverse event.

In one general aspect, the application describes a combination, such as a kit, comprising (a) a pharmaceutical composition, preferably a vaccine, comprising an adenoviral vector comprising a nucleic acid encoding an RSV F polypeptide that is stabilized in a pre-fusion conformation, wherein the effective amount of the pharmaceutical composition comprises about 1×10¹⁰ to about 1×10¹² viral particles of the adenoviral vector per dose, and (b) an influenza vaccine, preferably, a seasonal influenza vaccine. The combination can be used for inducing both a protective immune response against respiratory syncytial virus (RSV) infection and a protective immune response against influenza virus infection in a human subject in need thereof.

### BRIEF DESCRIPTION OF THE FIGURES

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a Forest plot of the geometric mean ratios of the hemagglutination inhibition (HI) antibody response (HAI) 28 days after vaccination for the per-protocol influenza immunogenicity population;

FIG. 2 shows a plot of the mean (95% CI) actual values over time of the HI antibody response (HAI) for the per-protocol influenza immunogenicity population;

FIG. 3 shows a Forest plot of the difference in seroconversion for the HI antibody response (HAI) 28 days after vaccination for the per-protocol influenza immunogenicity population;

FIG. 4 shows a Forest plot of the difference in seroprotection for the HI antibody response (HAI) 28 days after vaccination for the per-protocol influenza immunogenicity population;

FIG. 5 shows a plot of the titers of neutralizing antibodies to RSV A2 strain over time for the per-protocol RSV immunogenicity population, with geometric mean with 95% CI shown in the figure, and N=number of subjects with data at baseline;

FIG. 6 shows a plot of the antibody response by RSV pre-F protein, as measured by ELISA, over time, for the per-protocol RSV immunogenicity population, with geometric mean with 95% CI shown in the figure, and N=number of subjects with data at baseline;

FIG. 7 shows a plot of the antibody response by RSV post-F protein, as measured by ELISA, over time, for the per-protocol RSV immunogenicity population, with geometric mean with 95% CI shown in the figure, and N=number of subjects with data at baseline; and

FIG. 8 shows a box plot of RSV-F specific T cell response, as measured by IFN-γ ELISpot assay, over time, for the per-protocol RSV immunogenicity population.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/ characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The present invention provides methods for inducing both a protective immune response against respiratory syncytial virus (RSV) infection and a protective immune response against influenza virus in a human subject in need thereof, comprising intramuscularly administering to the subject (a) an effective amount of a pharmaceutical composition, preferably a vaccine, comprising an adenoviral vector comprising a nucleic acid encoding an RSV F polypeptide that is stabilized in a pre-fusion conformation, and (b) an effective amount of an influenza vaccine.

As used herein, the term “RSV fusion protein,” “RSV F protein,” “RSV fusion polypeptide” or “RSV F polypeptide” refers to a fusion (F) protein of any group, subgroup, isolate, type, or strain of respiratory syncytial virus (RSV). RSV exists as a single serotype having two antigenic subgroups, A and B. Examples of RSV F protein include, but are not limited to, RSV F from RSV A, e.g. RSV A1 F protein and RSV A2 F protein, and RSV F from RSV B, e.g. RSV B1 F protein and RSV B2 F protein. As used herein, the term “RSV F protein” includes proteins comprising mutations, e.g., point mutations, fragments, insertions, deletions and splice variants of full length wild type RSV F protein.

According to particular embodiments, the RSV F polypeptides that are stabilized in the pre-fusion conformation are derived from an RSV A strain. In certain embodiments the RSV F polypeptides are derived from the RSV A2 strain. RSV F polypeptides that are stabilized in the pre-fusion conformation that are useful in the invention are RSV F proteins having at least one mutation as compared to a wild type RSV F protein, in particular as compared to the RSV F protein having the amino acid sequence of SEQ ID NO: 1. According to particular embodiments, RSV F polypeptides that are stabilized in the pre-fusion conformation that are useful in the invention comprise at least one mutation selected from the group consisting of K66E, N671, I76V, S215P, K394R, S398L, D486N, D489N, and D489Y.

According to particular embodiments, the RSV F polypeptides that are stabilized in the pre-fusion conformation comprise at least one epitope that is recognized by a pre-fusion specific monoclonal antibody, e.g. CR9501. CR9501 comprises the binding regions of the antibodies referred to as 58C5 in WO2011/020079 and WO2012/006596, which binds specifically to RSV F protein in its pre-fusion conformation and not to the post-fusion conformation.

In particular embodiments, the RSV F polypeptides further comprise a heterologous trimerization domain linked to a truncated F1 domain, as described in WO2014/174018 and WO2014/202570. As used herein a “truncated” F1 domain refers to a F1 domain that is not a full length F1 domain, i.e. wherein either N-terminally or C-terminally one or more amino acid residues have been deleted. According to particular embodiments, at least the transmembrane domain and cytoplasmic tail are deleted to permit expression as a soluble ectodomain. In certain embodiments, the trimerization domain comprises SEQ ID NO: 2 and is linked to amino acid residue 513 of the RSV F1 domain, either directly or through a linker. In certain embodiments, the linker comprises the amino acid sequence SAIG (SEQ ID NO: 3).

Examples of RSV F proteins stabilized in a pre-fusion conformation include, but are not limited to those described in WO2014/174018, WO2014/202570 and WO 2017/174564, the contents of which are incorporated herein by reference.

According to particular embodiments, the RSV F protein comprises an amino acid sequence of SEQ ID NO: 4 or SEQ ID NO: 5, or an amino acid sequence that is at least 75%, 80%, 95%, 90% or 95% identical to the amino acid sequence of SEQ ID NO: 4 or SEQ ID NO: 5.

Examples of nucleic acid encoding RSV F protein stabilized in a pre-fusion conformation include SEQ ID NO: 6 and SEQ ID NO: 7. It is understood by a skilled person that numerous different nucleic acid molecules can encode the same polypeptide as a result of the degeneracy of the genetic code. It is also understood that skilled persons can, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleic acid molecule encoding an amino acid sequence” includes all nucleotide sequences that are degenerate versions of each other and that encode the same amino acid sequence. Nucleotide sequences that encode proteins and RNA can include introns. Sequences herein are provided from 5′ to 3′ direction, as custom in the art.

Influenza viruses are classified into influenza virus types: genus A, B and C. As used herein, the term “influenza virus” refers to any influenza virus type A, B or C, and any subtype therein. Influenza A virus variants are further characterized into subtypes by combinations of the hemagglutinin (H) and neuramidase (N) viral surface proteins. As used herein, the nomenclature for human influenza virus strains or isolates includes the type (genus) of virus, i.e. A, B or C and the geographical location of the first isolation, e.g., A/Michigan, A/Hong Kong, B/Brisbane, B/Phuket, etc.

As used herein, the term “vaccine” refers to a composition containing an active component effective to induce a certain degree of immunity in a subject against a certain pathogen or disease, which will result in at least a decrease, and up to complete absence, of the severity, duration or other manifestation of symptoms associated with infection by the pathogen or the disease. In the present invention, a vaccine comprises an adenovirus comprising a nucleic acid encoding an RSV F polypeptide that is stabilized in the pre-fusion conformation. According to embodiments of the application, a vaccine can be used to prevent serious lower respiratory tract disease leading to hospitalization and decrease the frequency of complications such as pneumonia and bronchiolitis due to RSV infection and replication in a subject. In certain embodiments, a vaccine can be a combination vaccine that further comprises other components that induce a protective immune response, e.g. against other proteins of RSV and/or against other infectious agents. The administration of further active components can for instance be done by separate administration or by administering combination products of the vaccines of the invention and the further active components.

In certain embodiments, the influenza vaccine is a seasonal influenza vaccine, which is defined as a vaccine directed against the seasonal occurring influenza viruses in a flu season. Examples of seasonal influenza vaccine include, but are not limited to, trivalent A/H1N1-A/H3N2 B vaccines. In certain embodiments, the seasonal influenza vaccine can be any commercially available seasonal influenza vaccine. Examples of commercially available seasonal influenza vaccine include, e.g., split vaccines BEGRIVAC™ (Wyath), FLUARIX™ (GSK), FLUZONE™ (Sanofi), and FLUSHIELD™ (Jamieson); subunit vaccines FLUVIRIN™ (Seqirus), AGRIPPAL™ (Novartis), and INFLUVAC™ (Abbott), and live attenuated influenza virus vaccine Flumist™ (Medimmune Inc.).

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with the agent.

As used herein, the term “induce” and variations thereof refers to any measurable increase in cellular activity. Induction of a protective immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

The ability to induce a protective immune response against RSV F protein and/or against influenza virus can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by methods readily known in the art, e.g., by measurement of cytokine profiles secreted by activated effector cells including those derived from CD4+ and CD8+ T-cells (e.g. quantification of IL-4 or IFN gamma-producing cells by ELISPOT), by measuring PBMC proliferation, by measuring NK cell activity, by determination of the activation status of immune effector cells (e.g. T-cell proliferation assays by a classical [3H] thymidine uptake), by assaying for antigen-specific T lymphocytes in a sensitized subject (e.g. peptide-specific lysis in a cytotoxicity assay, etc.). Additionally, IgG and IgA antibody secreting cells with homing markers for local sites which can indicate trafficking to the gut, lung and nasal tissues can be measured in the blood at various times after immunization as an indication of local immunity, and IgG and IgA antibodies in nasal secretions can be measured; Fc function of antibodies and measurement of antibody interactions with cells such as PMNs, macrophages, and NK cells or with the complement system can be characterized; and single cell RNA sequencing analysis can be used to analyze B cell and T cell repertoires.

The ability to induce a protective immune response against RSV F protein and/or against influenza virus can be determined by testing a biological sample (e.g., nasal wash, blood, plasma, serum, PBMCs, urine, saliva, feces, cerebral spinal fluid, bronchoalveolar lavage or lymph fluid) from the subject for the presence of antibodies, e.g. IgG or IgM antibodies, directed to the RSV F protein(s) administered in the composition, e.g. viral neutralizing antibody against RSV A2 (VNA A2), VNA RSV A Memphis 37b, RSV B, pre-F antibodies, post-F antibodies, RSV Ga antibodies, RSV Gb antibodies (see for example Harlow, 1989, Antibodies, Cold Spring Harbor Press), or, e.g., IgG or IgM antibodies directed to influenza virus protein(s) administered in the influenza virus vaccine, e.g., hemagglutination-inhibition (HI) or microneutralization (MN) antibodies. For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT, measurement of Fc interactions with complement, PMNs, macrophages and NK cells, with and without complement enhancement, or Antibody-Dependent Cellular Phagocytosis (ADCP) Assay. Exemplary methods are described in Example 1. According to particular embodiments, the induced immune response is characterized by neutralizing antibodies to RSV and/or protective immunity against RSV. According to particular embodiments, the induced immune response is characterized by neutralizing antibodies to influenza virus and/or protective immunity against influenza virus.

According to particular embodiments, the protective immune response is characterized by the presence of neutralizing antibodies to RSV and/or protective immunity against RSV, preferably detectable 8 to 35 days after administration of the pharmaceutical composition, such as 8, 9, 10, 11, 12, 13, 14, 15, 16, 17, 18, 19, 20, 21, 22, 23, 24, 25, 26, 27, 28, 29, 30, 31, 32, 33, 34 or 35 days after administration of the pharmaceutical composition. More preferably, the neutralizing antibodies against RSV are detected about 6 months to 5 years after the administration of the immunogenic components, such as 6 months, 1 year, 2 years, 3 years, 4 years or 5 years after administration of the immunogenic components.

According to particular embodiments, the protective immune response is characterized by the presence of neutralizing antibodies to influenza virus and/or protective immunity against influenza virus, preferably detectable 8 to 35 days after administration of the pharmaceutical composition, such as 8, 9, 10, 11, 12, 13, 14, 15, 16, 17, 18, 19, 20, 21, 22, 23, 24, 25, 26, 27, 28, 29, 30, 31, 32, 33, 34 or 35 days after administration of the pharmaceutical composition. More preferably, the neutralizing antibodies against influenza are detected about 6 months to 5 years after the administration of the immunogenic components, such as 6 months, 1 year, 2 years, 3 years, 4 years or 5 years after administration of the immunogenic components.

According to particular embodiments, the protective immune response that is induced against RSV upon co-administration of (a) and (b) is characterized by non-inferiority to the protective immune response that is induced against RSV upon administration of (a) alone. According to particular embodiments, the protective immune response that is induced against influenza virus upon co-administration of (a) and (b) is characterized by non-inferiority to the protective immune response that is induced against influenza virus upon administration of (b) alone. As used herein, non-inferiority is determined using a margin of 2 for the geometric mean titers (GMTs) of RSV-specific antibodies or influenza virus-specific antibodies. Exemplary methods are described in Example 1.

According to particular embodiments, the protective immune response is characterized by an absent or reduced RSV clinical symptom in the subject upon exposure to RSV. According to particular embodiments, the protective immune response is characterized by an absent or reduced influenza virus clinical symptom in the subject upon exposure to influenza virus. RSV and influenza clinical symptoms include, for example, upper respiratory symptoms including, e.g., runny nose, stuffy nose, sneezing, sore throat, earache; lower respiratory symptoms including, e.g., cough, shortness of breath, chest tightness, wheezing, sputum production; and systemic symptoms including, e.g., malaise, headache, muscle and/or joint ache, chilliness/feverishness.

As used herein, the term “adverse event” (AE) refers to any untoward medical occurrence in a patient administered a pharmaceutical product and which does not necessarily have a causal relationship with the treatment. According to embodiments of the invention,

AEs are rated on a 4-point scale of increasing severity using the following definitions: Mild (Garde 1): no interference with activity; Moderate (Grade 2): some interference with activity, not requiring medical intervention; Severe (Grade 3): prevents daily activity and requires medical intervention; Potentially life-threatening (Grade 4): symptoms causing inability to perform basis self-care functions OR medical or operative intervention indicated to prevent permanent impairment, persistent disability. A “severe adverse event,” “severe AE,” “SAE” can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; inpatient hospitalization, i.e., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that may jeopardize the patients or may require medical or surgical intervention to prevent one of the other outcomes listed above (e.g. intensive treatment in an emergency room or at home for allergic bronchospasm or blood dyscrasias or convulsions that do not result in hospitalization). Hospitalization is official admission to a hospital. Hospitalization or prolongation of a hospitalization constitutes criteria for an AE to be serious; however, it is not in itself considered an SAE. In the absence of an AE, hospitalization or prolongation of hospitalization is not considered an SAE. This can be the case, in the following situations: the hospitalization or prolongation of hospitalization is needed for a procedure required by the protocol; or the hospitalization or prolongation of hospitalization is a part of a routine procedure followed by the center (e.g. stent removal after surgery). Hospitalization for elective treatment of a pre-existing condition that did not worsen during the study is not considered an AE. Complications that occur during hospitalization are AEs. If a complication prolongs hospitalization, or meets any of the other SAE criteria, then the event is an SAE.

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease. For example, the effective amount of pharmaceutical composition also depends on whether adjuvant is also administered, with higher dosages being required in the absence of adjuvant.

According to embodiments of the application, an effective amount of pharmaceutical composition comprises an amount of pharmaceutical composition that is sufficient to induce a protective immune response against RSV F protein without inducing a severe adverse event. In particular embodiments, an effective amount of pharmaceutical composition comprises from about 1×10¹⁰ to about 1×10¹² viral particles per dose, preferably about 1×10¹¹ viral particles per dose, of an adenoviral vector comprising a nucleic acid encoding an RSV F polypeptide that is stabilized in a pre-fusion conformation.

According to embodiments of the application, an effective amount of pharmaceutical composition comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F polypeptide that is stabilized in a pre-fusion conformation. Preferably the recombinant RSV F polypeptide has an amino acid sequence of SEQ ID NO: 4 or SEQ ID NO: 5, and the adenoviral vector is of serotype 26, such as a recombinant Ad26.

According to embodiments of the application, an effective amount of influenza virus vaccine comprises an amount of influenza virus vaccine that is sufficient to induce a protective immune response against influenza virus without inducing a severe adverse event. In particular embodiments, an effective amount of influenza virus vaccine comprises a single dose of a commercially available seasonal influenza virus vaccine.

According to particular embodiments, the human subject is susceptible to RSV infection. In certain embodiments, a human subject that is susceptible to RSV infection includes, but is not limited to, an elderly human subject, for example a human subject ≥50 years old, ≥60 years old, preferably ≥65 years old; a young human subject, for example a human subject ≤5 years old, ≤1 year old; and/or a human subject that is hospitalized or a human subject that has been treated with an antiviral compound but has shown an inadequate antiviral response.

According to particular embodiments, the human subject is susceptible to influenza virus infection. In certain embodiments, a human subject that is susceptible to influenza virus infection includes, but is not limited to, an elderly human subject, for example a human subject ≥50 years old, ≥60 years old, preferably ≥65 years old; a young human subject, for example a human subject ≤5 years old, ≤1 year old; and/or a human subject that is hospitalized or a human subject that has been treated with an antiviral compound but has shown an inadequate antiviral response. In certain embodiments, a human subject that is susceptible to RSV infections includes, but is not limited to, a human subject with chronic heart disease, chronic lung disease, and/or immunodeficiencies.

According to particular embodiments, a human subject in need thereof is administered with a pharmaceutical composition comprising an adenovirus comprising a nucleic acid molecule encoding an RSV F polypeptide that is stabilized in the pre-fusion conformation and an influenza vaccine.

In certain embodiments, the adenovirus is a human recombinant adenovirus, also referred to as recombinant adenoviral vectors. The preparation of recombinant adenoviral vectors is well known in the art. The term “recombinant” for an adenovirus, as used herein implicates that it has been modified by the hand of man, e.g. it has altered terminal ends actively cloned therein and/or it comprises a heterologous gene, i.e. it is not a naturally occurring wild type adenovirus.

In certain embodiments, an adenoviral vector according to the invention is deficient in at least one essential gene function of the E1 region, e.g. the E1a region and/or the E1b region, of the adenoviral genome that is required for viral replication. In certain embodiments, an adenoviral vector according to the invention is deficient in at least part of the non-essential E3 region. In certain embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region).

Adenoviral vectors, methods for construction thereof and methods for propagating thereof, are well known in the art and are described in, for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913, and Thomas Shenk, “Adenoviridae and their Replication”, M. S.

Horwitz, “Adenoviruses”, Chapters 67 and 68, respectively, in Virology, B. N. Fields et al., eds., 3d ed., Raven Press, Ltd., New York (1996), and other references mentioned herein. Typically, construction of adenoviral vectors involves the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DN A, 2d ed., Scientific American Books (1992), and Ausubel et al., Current Protocols in Molecular Biology, Wiley Interscience Publishers, NY (1995), and other references mentioned herein.

In certain embodiments, the adenovirus is a human adenovirus of the serotype 26 or 35.

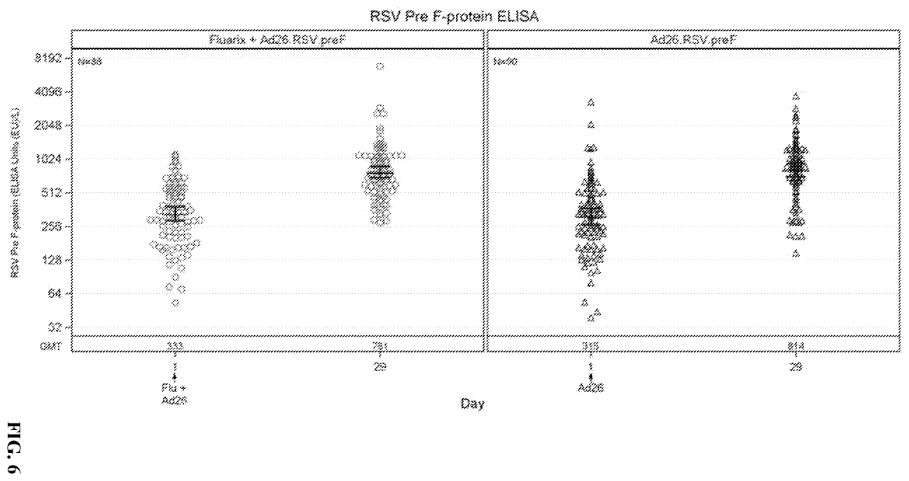

Preparation of rAd26 vectors is described, for example, in WO 2007/104792 and in Abbink et al., Virol. 2007:81(9): 4654-63. Exemplary genome sequences of Ad26 are found in GenBank Accession EF 153474 and in SEQ ID NO: 1 of WO 2007/104792. Preparation of rAd35 vectors is described, for example, in U.S. Pat. No. 7,270,811, in WO 00/70071, and in Vogels et al, J Virol. 2003:77(15): 8263-71. Exemplary genome sequences of Ad35 are found in GenBank Accession AC 000019 and in FIG. 6 of WO 00/70071.

A recombinant adenovirus according to the invention can be replication-competent or replication-deficient. In certain embodiments, the adenovirus is replication deficient, e.g. because it contains a deletion in the E1 region of the genome. As known to the skilled person, in case of deletions of essential regions from the adenovirus genome, the functions encoded by these regions have to be provided in trans, preferably by the producer cell, i.e. when parts or whole of E1, E2 and/or E4 regions are deleted from the adenovirus, these have to be present in the producer cell, for instance integrated in the genome thereof, or in the form of so-called helper adenovirus or helper plasmids. The adenovirus can also have a deletion in the E3 region, which is dispensable for replication, and hence such a deletion does not have to be complemented.

In certain embodiments, the adenovirus is a replication-incompetent adenovirus. According to particular embodiments, the adenovirus is a replication-incompetent Ad26 adenovirus. According to particular embodiments, the adenovirus is a replication-incompetent Ad35 adenovirus.

A producer cell (sometimes also referred to in the art and herein as “packaging cell” or “complementing cell” or “host cell”) that can be used can be any producer cell wherein a desired adenovirus can be propagated. For example, the propagation of recombinant adenovirus vectors is done in producer cells that complement deficiencies in the adenovirus. Such producer cells preferably have in their genome at least an adenovirus E1 sequence, and thereby are capable of complementing recombinant adenoviruses with a deletion in the E1 region. Any E1-complementing producer cell can be used, such as human retina cells immortalized by E1, e.g. 911 or PER.C6 cells (see U.S. Pat. No. 5,994,128), E1-transformed amniocytes (See EP patent 1230354), E1-transformed A549 cells (see e.g. WO 98/39411, U.S. Pat. No. 5,891,690), GH329:HeLa (Gao et al., Human Gene Therapy 2000:11 : 213-219), 293, and the like. In certain embodiments, the producer cells are for instance HEK293 cells, or PER.C6 cells, or 911 cells, or IT293SF cells, and the like.

For non-subgroup C E1-deficient adenoviruses such as Ad35 (subgroup B) or Ad26 (subgroup D), it is preferred to exchange the E4-orf6 coding sequence of these non-subgroup C adenoviruses with the E4-orf6 of an adenovirus of subgroup C such as Ad5. This allows propagation of such adenoviruses in well-known complementing cell lines that express the E1 genes of Ad5, such as for example 293 cells or PER.C6 cells (see, e.g. Havenga et al., J Gen. Virol. 2006:87: 2135-2143; WO 03/104467, incorporated in its entirety by reference herein). In certain embodiments, an adenovirus that can be used is a human adenovirus of serotype 35, with a deletion in the E1 region into which the nucleic acid encoding RSV F protein antigen has been cloned, and with an E4 orf6 region of Ad5. In certain embodiments, the adenovirus in the vaccine composition of the invention is a human adenovirus of serotype 26, with a deletion in the E1 region into which the nucleic acid encoding RSV F protein antigen has been cloned, and with an E4 orf6 region of Ad5.

In alternative embodiments, there is no need to place a heterologous E4orf6 region (e.g. of Ad5) in the adenoviral vector, but instead the E1-deficient non- subgroup C vector is propagated in a cell line that expresses both E1 and a compatible E4orf6, e.g. the 293-ORF6 cell line that expresses both E1 and E4orf6 from Ad5 (see e.g. Brough et al, J Virol. 1996:70: 6497-501 describing the generation of the 293-ORF6 cells; Abrahamsen et al, J Virol. 1997:71 : 8946-51 and Nan et al, Gene Therapy 2003:10: 326-36 each describing generation of E1 deleted non-subgroup C adenoviral vectors using such a cell line).

Alternatively, a complementing cell that expresses E1 from the serotype that is to be propagated can be used (see e.g. WO 00/70071, WO 02/40665).

For subgroup B adenoviruses, such as Ad35, having a deletion in the E1 region, it is preferred to retain the 3′ end of the E1B 55K open reading frame in the adenovirus, for instance the 166 bp directly upstream of the pIX open reading frame or a fragment comprising this such as a 243 bp fragment directly upstream of the pIX start codon (marked at the 5′ end by a Bsu361 restriction site in the Ad35 genome), since this increases the stability of the adenovirus because the promoter of the pIX gene is partly residing in this area (see, e.g. Havenga et al, 2006, J. Gen. Virol. 87: 2135-2143; WO 2004/001032, incorporated by reference herein).

Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

According to particular embodiments, a pharmaceutical composition useful for the invention further comprises a pharmaceutically acceptable carrier or excipient. As used herein, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Science (15th ed.), Mack Publishing Company, Easton, Pa., 1980). The preferred formulation of the pharmaceutical composition depends on the intended mode of administration and therapeutic application. The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or non-toxic, non-therapeutic, non-immunogenic stabilizers, and the like. It will be understood that the characteristics of the carrier, excipient or diluent will depend on the route of administration for a particular application.

In some embodiments, the pharmaceutically acceptable carrier comprises one or more salts, such as sodium chloride, potassium chloride, magnesium chloride, one or more amino acids, such as arginine, glycine, histidine and/or methionine, one or more carbohydrates, such as lactose, maltose, sucrose, one or more surfactants, such as polysorbate 20, polysorbate 80, one or more chelators, such as ethylenediaminetetracetic acid (EDTA), and ethylenediamine-N,N′-disuccinic acid (EDDS), and one or more alcohols such as ethanol and methanol. Preferably, the pharmaceutical composition has a pH of 5 to 8, such as a pH of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or any value in between.

In some embodiments, a pharmaceutical composition for use in the invention comprises sodium chloride, potassium chloride, and/or magnesium chloride at a concentration of 1 mM to 100 mM, 25 mM to 100 mM, 50 mM to 100 mM, or 75 mM to 100 mM. For example, the concentration of sodium chloride, potassium chloride, and/or magnesium chloride can be 1 mM, 5 mM, 10 mM, 15 mM, 20 mM, 25 mM, 30 mM, 35 mM, 40 mM, 45 mM, 50 mM, 55mM, 60 mM, 65 mM, 70 mM, 75 mM, 80 mM, 85 mM, 90 mM, 95 mM, 100mM, or any concentration in between.

In some embodiments, a pharmaceutical composition for use in the invention comprises histidine, arginine, and/or glycine at a concentration of 1 mM to 50 mM, 5 mM to 50 mM, 5 mM to 30 mM, 5 mM to 20 mM, or 10 mM to 20 mM. For example, the concentration of histidine, arginine, and/or glycine can be 1 mM, 2 mM 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM or 50 mM, or any concentration in between.

In some embodiments, a pharmaceutical composition for use in the invention comprises sucrose, lactose, and/or maltose at a concentration of 1% to 10% weight by volume (w/v) or 5% to 10% (w/v). For example, the concentration of sucrose, lactose, and/or maltose can be 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

In some embodiments, a pharmaceutical composition for use in the invention comprises polysorbate 20 (PS20) and/or polysorbate 80 (PS80) at a concentration of 0.01% (w/v) to 0.1% (w/v), 0.01% (w/v) to 0.08% (w/v), or 0.02% (w/v) to 0.05% (w/v). For example, the concentration of polysorbate 20 and/or polysorbate 80 can be 0.01%, 0.02%, 0.03%, 0.04%, 0.05%, 0.06%, 0.07%, 0.08%, 0.09% or 0.1% (w/v), or any concentration in between.

In some embodiments, a pharmaceutical composition for use in the invention comprises ethylenediaminetetracetic acid (EDTA) and/or ethylenediamine-N,N′-disuccinic acid (EDDS) at a concentration of 0.1 mM to 5 mM, 0.1 mM to 2.5 mM, or 0.1 to 1 mM. For example, the concentration of EDTA and/or EDDS can be 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM, or any concentration in between.

In some embodiments, a pharmaceutical composition for use in the invention comprises ethanol and/or methanol at a concentration of 0.1% to 5% weight by volume (w/v) or 0.5% to 5% (w/v). For example, the concentration of ethanol and/or methanol can be 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 1.5% (w/v), 2% (w/v), 2.5% (w/v), 3% (w/v), 3.5% (w/v), 4% (w/v), 4.5% (w/v), or 5% (w/v), or any concentration in between.

Pharmaceutical compositions comprising an adenovirus comprising a nucleic acid molecule encoding an RSV F polypeptide that is stabilized in the pre-fusion conformation for use in the invention can be prepared by any method known in the art in view of the present disclosure. For example, an adenovirus comprising a nucleic acid molecule encoding an RSV F polypeptide that is stabilized in the pre-fusion conformation can be mixed with one or more pharmaceutically acceptable carriers to obtain a solution. The solution can be stored as a frozen liquid at a controlled temperature ranging from −55° C.±10° C. to −85° C.±10° C. in an appropriate vial until administered to the subject.

In certain embodiments, pharmaceutical compositions according to the invention further comprise one or more adjuvants. Adjuvants are known in the art to further increase the immune response to an applied antigenic determinant. The terms “adjuvant” and “immune stimulant” are used interchangeably herein, and are defined as one or more substances that cause stimulation of the immune system. In this context, an adjuvant is used to enhance a protective immune response to the RSV F polypeptides of the pharmaceutical compositions of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc.), which stimulate immune response upon interaction with recipient cells. In certain embodiments the pharmaceutical compositions of the invention comprise aluminium as an adjuvant, e.g. in the form of aluminium hydroxide, aluminium phosphate, aluminium potassium phosphate, or combinations thereof, in concentrations of 0.05-5 mg, e.g. 0.075-1.0 mg, of aluminium content per dose.

According to particular embodiments, a pharmaceutical composition comprising an adenoviral vector comprising a nucleic acid encoding an RSV F polypeptide stabilized in a pre-fusion conformation is used in combination with an influenza vaccine, such as a seasonal influenza vaccine. Preferably, the pharmaceutical composition and the influenza vaccine are co-administered.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a pharmaceutical composition described herein) can be administered prior to (e.g., 1 minutes, 2 minutes, 3 minutes, 4 minutes, 5 minutes, 10 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minutes, 2 minutes, 3 minutes, 4 minutes, 5 minutes, 10 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, the term “co-administered,” in the context of the administration of two or more therapies to a subject, refers to the use of the two or more therapies in combination and the two or more therapies are administered to the subject within a period of 24 hours. In certain embodiments, “co-administered” therapies are pre-mixed and administered to a subject together at the same time. In other embodiments, “co-administered” therapies are administered to a subject in separate compositions within 24 hours, such as within 12 hours, 10 hours, 8 hours, 6 hours, 4 hours, 2 hours, 1 hour or less. Preferably, “co-administered” therapies are administered to a subject in separate compositions within 60 minutes, such as within 30 minutes, 20 minutes, 10 minutes, 5 minutes or less. “Co-administered” therapies are administered to a subject in separate compositions at the same time.

The timing of administrations can vary significantly from once a day, to once a year, to once a decade. A typical regimen consists of an immunization followed by booster injections at time intervals, such as 1 to 24 week intervals. Another regimen consists of an immunization followed by booster injections 1, 2, 4, 6, 8, 10 and 12 months later. Another regimen entails an injection every two months for life. Another regimen entails an injection every year or every 2, 3, 4 or 5 years. Alternatively, booster injections can be on an irregular basis as indicated by monitoring of immune response.

It is readily appreciated by those skilled in the art that the regimen for the priming and boosting administrations can be adjusted based on the measured immune responses after the administrations. For example, the boosting compositions are generally administered weeks or months after administration of the priming composition, for example, about 1 week, or 2-3 weeks or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two years after administration of the priming composition.

According to particular aspects, one or more boosting immunizations can be administered. The antigens in the respective priming and boosting compositions, however many boosting compositions are employed, need not be identical, but should share antigenic determinants or be substantially similar to each other.

Pharmaceutical compositions of the present invention can be formulated according to methods known in the art in view of the present disclosure.

The pharmaceutical compositions can be administered by suitable means for prophylactic and/or therapeutic treatment. Non-limiting embodiments include parenteral administration, such as intradermal, intramuscular, subcutaneous, transcutaneous, or mucosal administration, e.g. intranasal, oral, and the like. In one embodiment, a composition is administered by intramuscular injection. The skilled person knows the various possibilities to administer a pharmaceutical composition in order to induce an immune response to the antigen(s) in the pharmaceutical composition. In certain embodiments, a composition of the invention is administered intramuscularly.

The invention also provides methods for vaccinating a subject against both RSV infection and influenza virus infection without inducing a severe adverse effect in a human subject in need thereof. In particular embodiments, the method comprises administering to the subject (a) an effective amount of a pharmaceutical composition, preferably a vaccine, comprising an adenoviral vector comprising a nucleic acid encoding an RSV F polypeptide that is stabilized in a pre-fusion conformation, and (b) an effective amount of an influenza vaccine.

According to embodiments of the application, an effective amount of pharmaceutical composition comprises an amount of pharmaceutical composition that is sufficient to vaccinate a subject against RSV infection without inducing a severe adverse event. In particular embodiments, an effective amount of pharmaceutical composition comprises from about 1×10¹⁰ to about 1×10¹² viral particles per dose, preferably about 1×10¹¹ viral particles per dose, of an adenoviral vector comprising a nucleic acid encoding an RSV F polypeptide that is stabilized in a pre-fusion conformation.

According to embodiments of the application, an effective amount of pharmaceutical composition comprises about 1×10¹⁰ to about 1×10¹² viral particles per dose, such as about 1×10¹⁰ viral particles per dose, about 2×10¹⁰ viral particles per dose, about 3×10¹⁰ viral particles per dose, about 4×10¹⁰ viral particles per dose, about 5×10¹⁰ viral particles per dose, about 6×10¹⁰ viral particles per dose, about 7×10¹⁰ viral particles per dose, about 8×10¹⁰ viral particles per dose, about 9×10¹⁰ viral particles per dose, about 1×10¹¹ viral particles per dose, about 2×10¹¹ viral particles per dose, about 3×10¹¹ viral particles per dose, about 4×10¹¹ viral particles per dose, about 5×10¹¹ viral particles per dose, about 6×10¹¹ viral particles per dose, about 7×10¹¹ viral particles per dose, about 8×10¹¹ viral particles per dose, about 9×10¹¹ viral particles per dose, or about 1×10¹² viral particles per dose, of an adenoviral vector comprising a nucleic acid encoding an RSV F polypeptide that is stabilized in a pre-fusion conformation. Preferably the recombinant RSV F polypeptide has an amino acid sequence of SEQ ID NO: 4 or SEQ ID NO: 5, and the adenoviral vector is of serotype 26, such as a recombinant Ad26.

According to embodiments of the application, an effective amount of influenza virus vaccine comprises an amount of influenza virus vaccine that is sufficient to induce a protective immune response against influenza virus without inducing a severe adverse event. In particular embodiments, an effective amount of influenza virus vaccine comprises a single dose of a commercially available seasonal influenza virus vaccine.

### EXAMPLES

The following examples of the invention are to further illustrate the nature of the invention. It should be understood that the following examples do not limit the invention and that the scope of the invention is to be determined by the appended claims.

### Example 1: Phase 2a Human Study

A Phase 2a, randomized, double-blind, placebo-controlled study was carried out to evaluate the safety and immunogenicity of seasonal influenza vaccine and Ad26.RSV.preF, a replication-incompetent Ad26 containing a DNA transgene that encodes for a pre-fusion conformation-stabilized F protein (pre-F) of a RSV A2 strain, with and without co-administration, in healthy adults aged 60 years and older.

Study Design/Overview—

A single center, randomized, placebo-controlled, double-blind Phase 2a study was conducted in approximately 180 adult male and female subjects ≥60 years of age in stable health randomized in parallel in a 1:1 ratio to one of two groups:
Group 1 (co-administered (“CoAd”)) received 1×10¹¹ viral particles (vp) of Ad26.RSV.preF on Day 1 administered at the same time as a commercially available seasonal influenza vaccine (fluarix), and placebo on Day 29.
Group 2 (Control) received placebo on Day 1, administered at the same time as a commercially available seasonal influenza vaccine (fluarix), and 1×10¹¹ vp Ad26.RSV.preF on Day 29.

All study vaccines were given intramuscularly, and a schematic overview of the study design and groups is depicted in Table 1 below:

TABLE 1

| Vaccine group | Day 1 | Day 29 |
|---|---|---|
| Group 1 | Ad26.RSV.preF (1 × 10¹¹ | Placebo |
| (N = 90) | vp) + Fluarix | |
| Group 2 | Placebo + | Ad26.RSV.preF |
| (N = 90) | Fluarix | (1 × 10¹¹ vp) |

N = number of subjects

Vaccination Schedules/Study duration: The study duration was about 30 weeks per participant, and the study consisted of vaccinations on Day 1 and Day 29, a 28- day follow-up period after each vaccination, and a follow-up until 6 months after the second vaccination. Solicited adverse events (AEs) were recorded 7 days after each vaccination. Unsolicited AEs were collected from informed consent forms until day 28 after each vaccination, and SAEs were assessed throughout the study. Immune responses were assessed on Day 1, 29 and 57.

Primary efficacy endpoint: The primary objectives of the study were (1) to assess non-inferiority of the concomitant administration of Ad26.RSV.preF and seasonal influenza vaccine versus the administration of seasonal influenza vaccine alone in terms of humoral immune response expressed by the geometric mean titers (GMTs) of hemagglutination inhibition (HI) antibody against all four influenza vaccine strains 28 days after the administration of influenza vaccine, using a non-inferiority margin of 2 for the GMT ratio (control group/co-administration group), and (2) to assess the safety and tolerability of a single dose of 1×10¹¹ vp Ad26.RSV.preF, administered intramuscularly to subjects aged ≥60 years, separately or concomitantly with the seasonal influenza vaccine.

Statistical method: The primary immunogenicity objective was assessed by calculating the 95% one-sided upper confidence limit for the difference in log-transformed HI antibody titers for each of the four seasonal influenza vaccine strains between Control (Group 2) and CoAd (Group 1) groups, using an analysis of variance (ANOVA) model with the Day 28 titer as dependent variable and regimen as covariate. The confidence limit was calculated using Welch-Satterthwaite t-interval method to allow for the estimation of separate variances per regimen. The confidence limit was back-transformed (by exponentiation) to a GMT ratio and compared to the non-inferiority limit of 2.

Results—

A total of 180 subjects were randomized and vaccinated, 90 subjects per group. Two subjects in the CoAd group (Group 1) discontinued the study before having received the second dose (reasons: refused further study treatment (1 subject) and discontinued due to AE (ear infection) (1 subject)). In addition, two more subjects (one in each group) discontinued the study after having received both doses (reason: lost to follow-up).

The primary analysis was performed after all subjects completed the safety and immunogenicity assessments on Day 57 (i.e., 28 days post-second dose). All data up to Day 57 were included in the analysis.

1. Primary Immunogenicity Analysis:

The immunogenicity analysis was based on the Per-protocol Influenza Immunogenicity (PPII) population, which is defined as all subjects who were randomized and received the first vaccination for whom immunogenicity data was available, excluding subjects with major protocol deviations expecting to impact the immunogenicity outcomes. Samples taken after a natural influenza infection were not included in the assessment of the immunogenicity of the seasonal influenza vaccine.

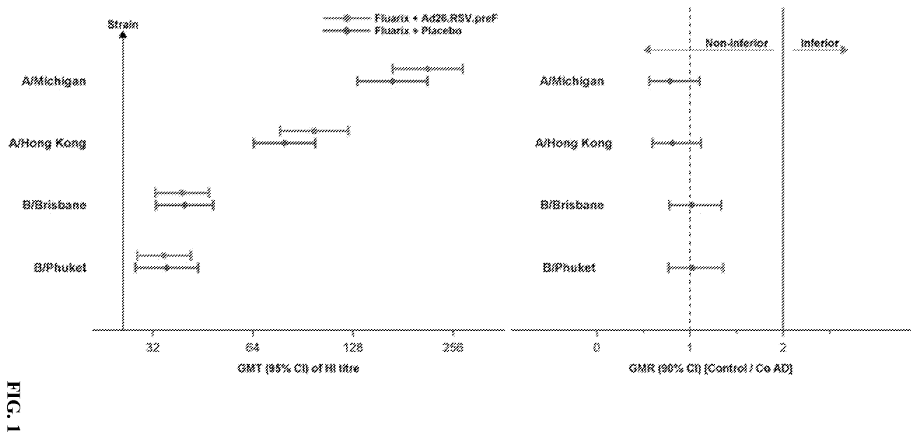

The geometric mean hemagglutination inhibition (HI) antibody titers (GMT) 28 days after vaccination with Fluarix, the geometric mean ratios (GMR) of the control group (Fluarix+Placebo) over the co-administered group (Fluarix+Ad26.RSV.preF), and their corresponding CIs for each of the four influenza strains are shown in Table 2 and FIG. 1. The upper confidence limits of the all four GMRs (Control group/CoAd group) were below the non-inferiority margin of 2. Hence, non-inferiority of co-administration of Ad26.RSV.preF+Fluarix versus the control group (Fluarix+Placebo) was concluded.

TABLE 2
HI Antibody Response 28 Days After Influenza Vaccination; PPII population

| | LS Means (95% CI) ^{#} | | | |
|---|---|---|---|---|
| Influenza Vaccine | Fluarix + | Fluarix + | Geometric Mean Ratio | |
| Strains | Ad26.RSV.preF | Placebo | (90% CI) ^{†} | P-value ^{†} |
| A/Michigan | 214.6 (159.66; 288.51) | 168.1 (125.15; 225.83) | 0.8 (0.55; 1.11) | <.001 |
| A/Hong Kong | 97.8 (73.29; 130.50) | 79.6 (61.32; 103.28) | 0.8 (0.59; 1.12) | <.001 |
| B/Brisbane | 39.2 (31.21; 49.14) | 39.8 (31.26; 50.75) | 1.0 (0.77; 1.34) | <.001 |
| B/Phuket | 34.5 (27.51; 43.38) | 35.2 (27.03; 45.93) | 1.0 (0.76; 1.36) | <.001 |

^{†} Based on Welch-Satterthwaite t-interval method. The difference (Control group minus CoAd group) and CI in log- transformed HI antibody titers were calculated for each of the four influenza vaccine strains, and were back-transformed (by exponentiation) to a GMT ratio (Control group/CoAd group) and the corresponding CI.
^{#} Least squares (LS) means of the log-transformed HI antibody titers, back-transformed (by exponentiation) to a GMT. The p-value is calculated based on a one-tailed t-test with alternative hypothesis: GMT ratio < 2.

Sensitivity analyses of the above non-inferiority analysis were conducted, once by adjusting for baseline HI levels in the model above and once running the model on the FA set. The results of the sensitivity analyses were in line with the above results.

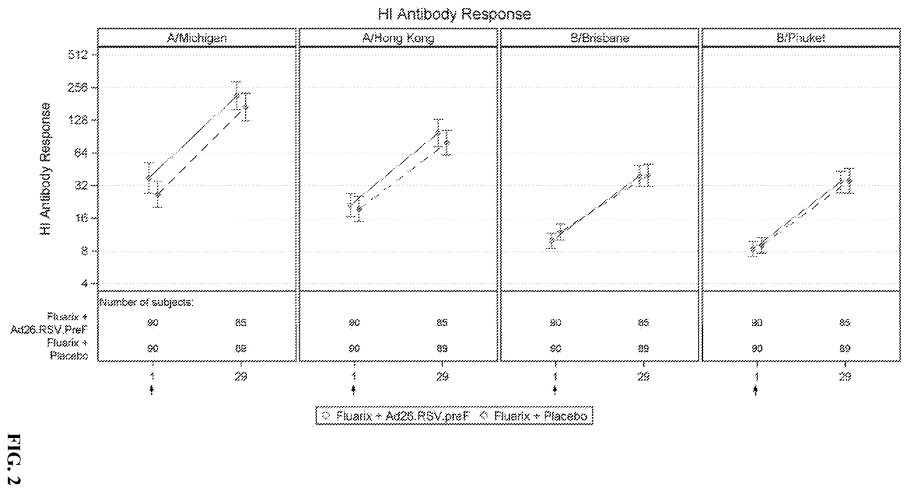

FIG. 2 shows a plot of the mean (95% CI) actual values over time of the HI antibody response (HAI) for the per-protocol influenza immunogenicity population.

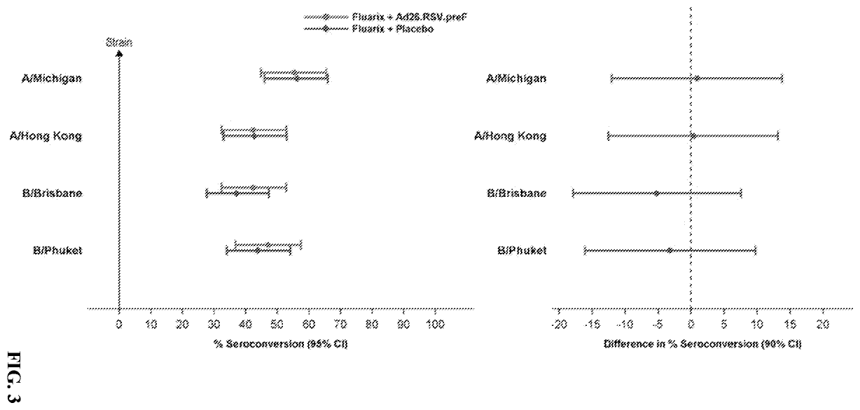

FIG. 3 shows a Forest plot of the difference in seroconversion for the HI antibody response (HAI) 28 days after vaccination for the per-protocol influenza immunogenicity population. Seroconversion rates against the four influenza vaccine strains was defined as a post-vaccination titer ≥1:40 in subjects with a pre-vaccination titer of <1:10, or a ≥4-fold titer increase in subjects with a pre-vaccination titer of ≥1:10. The difference in proportions of seroconverted subjects between groups (Control minus CoAd) and the 90% 2-sided CI were calculated based on the Wilson score method.

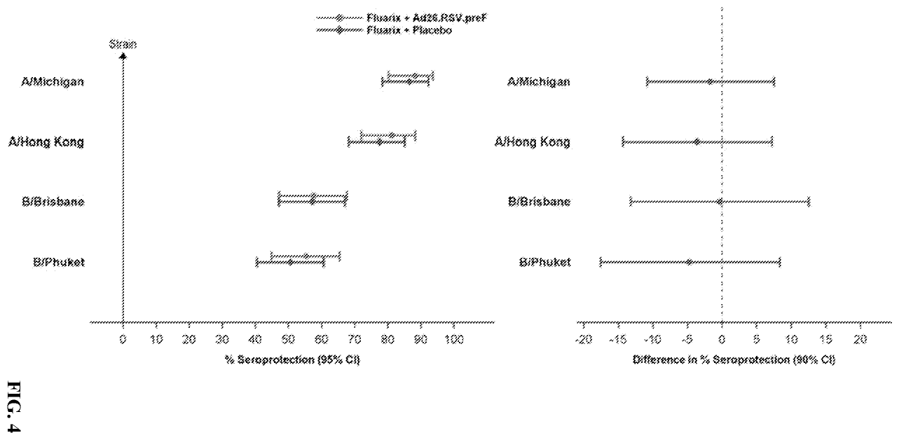

FIG. 4 shows a Forest plot of the difference in seroprotection for the HI antibody response (HAI) 28 days after vaccination for the per-protocol influenza immunogenicity population. Seroprotection rates against the four influenza vaccine strains was defined as the percentage of subjects with a post-vaccination titer ≥1:40. The difference in proportions of seroprotected subjects between groups (Control minus CoAd) and the 90% 2-sided CI were calculated based on the Wilson score method.

2. Secondary Immunogenicity Analysis:

The humeral immunogenicity analysis was based on the per-protocol RSV immunogenicity (PPRI) population, which is defined as all randomized and fully vaccinated subjects (all three vaccinations, i.e., seasonal influenza, Ad26.RSV.preF and placebo) for whom immunogenicity data were available, excluding subjects with major protocol deviations expecting to impact the immunogenicity outcomes. Samples taken after a natural RSV infection were not included in the assessment of the immunogenicity of Ad26.RSV.preF.

To assess the effect of co-administration of Ad26.RSV.preF with Fluarix on the viral neutralizing antibody against RSV A2 (VNA A2) levels, the GMT ratio of VNA A2 levels 28 days post dosing of the Ad26.RSV.preF alone vs the coadministration was calculated. This ratio with corresponding 90% CI was 1.2 (1.00; 1.45). The geometric mean VNA A2 titers (GMT) 28 days after Ad26.RSV.preF vaccination, the geometric mean ratios (GMR) of the control group (Ad26.RSV.preF) over the co-administered group (Fluarix+Ad26.RSV.preF), and the corresponding CIs are shown in Table 3.

TABLE 3
Neutralizing Antibodies to RSV A2 strain 28 Days
After Ad26.RSV.preF Vaccination; PPRI population

| | LS Means (95% CI) ^{#} | | |
|---|---|---|---|
| RSV | Fluarix + | | Geometric Mean Ratio |
| Antibody | Ad26.RSV.preF | Ad26.RSV.preF | (90% CI) ^{†} |
| VNA A2 | 1404.2 (1207.46; 1633.04) | 1689.7 (1432.69; 1992.74) | 1.2 (1.00; 1.45) |

^{†} Based on Welch-Satterthwaite t-interval method. The difference (Control group minus CoAd group) and CI in log- transformed HI antibody titers were calculated and were back-transformed (by exponentiation) to a GMT ratio (Control group/CoAd group) and the corresponding CI.
^{#} Least squares (LS) means of the log-transformed HI antibody titers, back-transformed (by exponentiation) to a GMT.

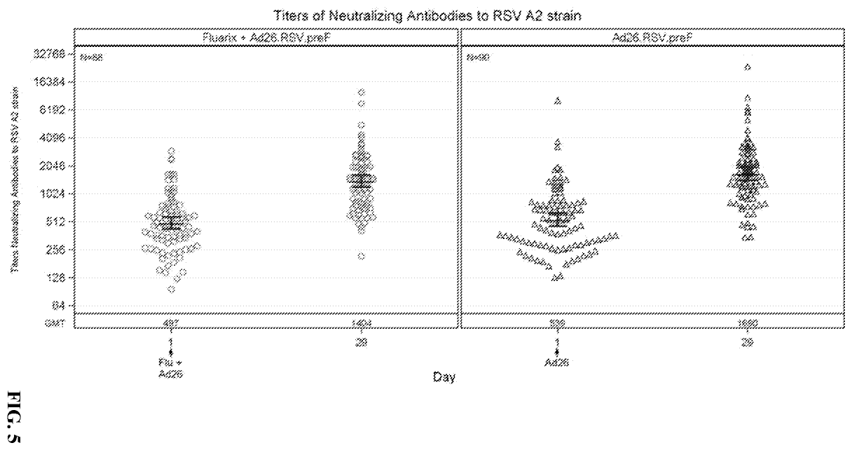

FIG. 5 shows a plot of the titers of neutralizing antibodies to RSV A2 strain over time for the per-protocol RSV immunogenicity population. The geometric mean of the fold rise and 95% CI of VNA A2 were 2.8 (2.5; 3.2) and 3.1 (2.7; 3.6) for the Fluarix+Ad26.RSV.preF arm and Ad26.RSV.preF alone arm, respectively.

FIG. 6 shows a plot of the antibody response by RSV pre-F protein, as measured by ELISA, over time, for the per-protocol RSV immunogenicity population, with geometric mean with 95% CI shown in the figure, and N=number of subjects with data at baseline. The geometric mean of the fold rise and 95% CI of pre-F ELISA were 2.3 (2.1; 2.7) and 2.6 (2.3; 3.0) for the Fluarix+Ad26.RSV.preF arm and the Ad26.RSV.preF alone arm, respectively.

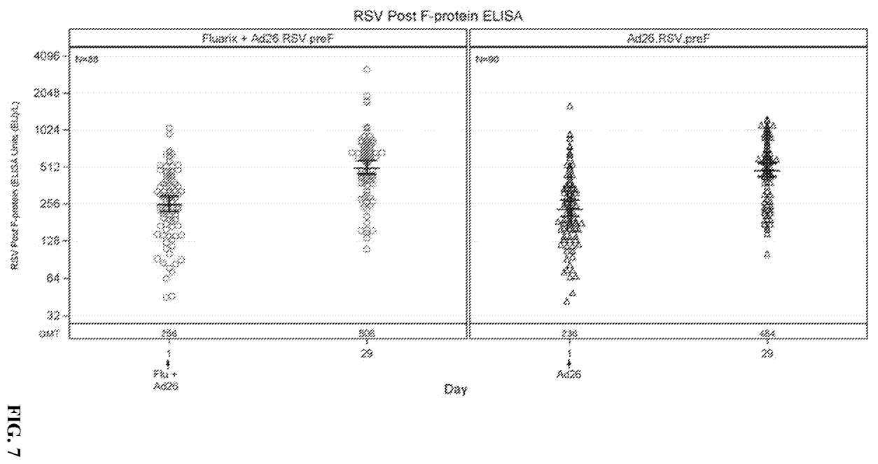

FIG. 7 shows a plot of the antibody response by RSV post-F protein, as measured by ELISA, over time, for the per-protocol RSV immunogenicity population, with geometric mean with 95% CI shown in the figure, and N=number of subjects with data at baseline. The geometric mean of the fold rise and 95% CI of post-F ELISA were 2.0 (1.8; 2.2) and 2.1 (1.9; 2.3) for the Fluarix+Ad26.RSV.preF arm and the Ad26.RSV.preF alone arm, respectively.

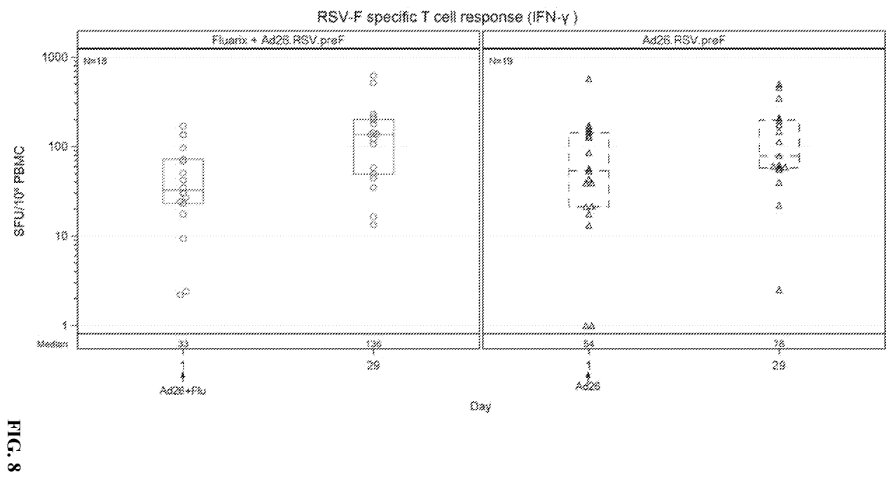

FIG. 8 shows a box plot of RSV-F specific T cell response, as measured by IFN-γ ELISpot assay, over time, for the per-protocol RSV immunogenicity population. Note that for two subjects, Day 29 ELISpot samples were omitted due to reconciliation/merging issues.

3. Safety:

The safety analysis was based on the full analysis (FA) population, which is defined as all subjects who were randomized and received at least one dose of study vaccine, regardless of the occurrence of protocol deviations and vaccine type.

One subject (1.1%) of group 1 (CoAd) reported 3 SAEs after Placebo dosing. The SAEs were Grade 4 hypertensive emergency, grade 4 Bradycardia and grade 3 renal injury. These AEs were considered not related to vaccination. No other SAEs were reported. There were no AEs with fatal outcome.

One subject (1.1%) of group 1 (CoAd) experienced an AE leading to discontinuation after Fluarix+Ad26.RSV.preF administration. This AE was a grade 2 ear infection, considered not related to vaccination.

The most frequently reported solicited local event was pain/tenderness, reported in the Ad26.RSV.preF arms in 78.9% or 76.7% of the subjects after Ad26.RSV.preF dosing with or without concomitant Fluarix dosing, respectively. In the Fluarix arms this was reported in 46.7% and 38.9% of the subjects after Fluarix dosing with or without concomitant Ad26.RSV.preF dosing, respectively. In the placebo arm, pain/tenderness was reported in less than 20%. The median time to onset for pain/tenderness was 1 day, the median duration was 2 or 4 days in the Ad26.RSV.preF arms when co-administered with Fluarix or not, and the median duration was 1 or 2 days in the placebo or Fluarix arms.

Solicited systemic AEs reported in more than 30% of the subjects after Ad26.RSV.preF dosing with or without Fluarix coadministration were arthralgia, chills, fatigue, headache and myalgia. These AEs were reported in less than 20% of the subjects after Fluarix alone administration or Placebo. In the groups with Ad26.RSV.preF vaccination (with or without Fluarix), the median time to onset was typically 1 to 2 days, and the median duration was in general 1 to 2 days.

Unsolicited AEs reported in more than 5% of the subjects after dosing were respiratory tract infection and increased blood pressure. Respiratory tract infection was reported in 12.2% of the subjects after Fluarix+Placebo dosing, in 11.1% of the subjects after Fluarix+Ad26.RSV.preF co-administration, in 5.6% of the subjects after Ad26.RSV.preF alone dosing, and in 8.0% of subjects after Placebo dosing.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

SEQUENCES
(RSV F protein A2 full length sequence)
SEQ ID NO: 1
MELLILKANAITTILTAVTFCFASGQNITEEFYQSTCSAVSKGYLSALRTGWYTSVITIE
LSNIKKNKCNGTDAKIKLIKQELDKYKNAVTELQLLMQSTPATNNRARRELPRFMN
YTLNNAKKTNVTLSKKRKRRFLGFLLGVGSAIASGVAVSKVLHLEGEVNKIKSALLS
TNKAVVSLSNGVSVLTSKVLDLKNYIDKQLLPIVNKQSCSISNIETVIEFQQKNNRLLE
ITREFSVNAGVTTPVSTYMLTNSELLSLINDMPITNDQKKLMSNNVQIVRQQSYSIMSI
IKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSV
ITSLGAIVSCYGKTKCTASNKNRGIIKTFSNGCDYVSNKGVDTVSVGNTLYYVNKQE
GKSLYVKGEPIINFYDPLVFPSDEFDASISQVNEKINQSLAFIRKSDELLHNVNAVKST
TNIMITTIIIVIIVILLSLIAVGLLLYCKARSTPVTLSKDQLSGINNIAFSN
(Trimerization domain)
SEQ ID NO: 2
GYIPEAPRDGQAYVRKDGEWVLLSTFL
(Linker)
SEQ ID NO: 3
SAIG
(RSV preF2.1)
SEQ ID NO: 4
MELLILKANAITTILTAVTFCFASGQNITEEFYQSTCSAVSKGYLGALRTGWYTSVITI
ELSNIKEIKCNGTDAKVKLIKQELDKYKNAVTELQLLMQSTPATNNRARRELPRFMN
YTLNNAKKTNVTLSKKRKRRFLGFLLGVGSAIASGVAVSKVLHLEGEVNKIKSALLS
TNKAVVSLSNGVSVLTSKVLDLKNYIDKQLLPIVNKQSCSIPNIETVIEFQQKNNRLLE
ITREFSVNAGVTTPVSTYMLTNSELLSLINDMPITNDQKKLMSNNVQIVRQQSYSIMSI
IKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSV
ITSLGAIVSCYGKTKCTASNKNRGIIKTFSNGCDYVSNKGVDTVSVGNTLYYVNKQE
GKSLYVKGEPIINFYDPLVFPSDEFDASISQVNEKINQSLAFIRKSDELLHNVNAVKST
TNIMITTIIIVIIVILLSLIAVGLLLYCKARSTPVTLSKDQLSGINNIAFSN
(RSV preF2.2)
SEQ ID NO: 5
MELLILKANAITTILTAVTFCFASGQNITEEFYQSTCSAVSKGYLSALRTGWYTSVITIE
LSNIKEiKCNGTDAKVKLIKQELDKYKNAVTELQLLMQSTPATNNRARRELPRFMNY
TLNNAKKTNVTLSKKRKRRFLGFLLGVGSAIASGVAVSKVLHLEGEVNKIKSALLST
NKAVVSLSNGVSVLTSKVLDLKNYIDKQLLPIVNKQSCSIPNIETVIEFQQKNNRLLEI
TREFSVNAGVTTPVSTYMLTNSELLSLINDMPITNDQKKLMSNNVQIVRQQSYSIMSII
KEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSV
ITSLGAIVSCYGKTKCTASNKNRGIIKTFSNGCDYVSNKGVDTVSVGNTLYYVNKQE
GKSLYVKGEPIINFYDPLVFPSNEFDASISQVNEKINQSLAFIRKSDELLHNVNAVKST
TNIMITTIIIVIIVILLSLIAVGLLLYCKARSTPVTLSKDQLSGINNIAFSN
(RSV F pre-F2.1)
SEQ ID NO: 6
ATGGAGCTGCTGATCCTGAAGGCCAACGCCATCACCACCATCCTGACCGCCGTG
ACCTTCTGCTTCGCCAGCGGCCAGAACATCACCGAGGAGTTCTACCAGAGCACCT
GCAGCGCCGTGAGCAAGGGCTACCTGGGCGCCCTGAGAACCGGCTGGTACACCA
GCGTGATCACCATCGAGCTGAGCAACATCAAGGAGATCAAGTGCAACGGCACCG
ACGCCAAGGTGAAGCTGATCAAGCAGGAGCTGGACAAGTACAAGAACGCCGTG
ACCGAGCTGCAGCTGCTGATGCAGAGCACCCCCGCCACCAACAACAGAGCCAGA
AGAGAGCTGCCCAGATTCATGAACTACACCCTGAACAACGCCAAGAAGACCAAC
GTGACCCTGAGCAAGAAGAGAAAGAGAAGATTCCTGGGCTTCCTGCTGGGCGTG
GGCAGCGCCATCGCCAGCGGCGTGGCCGTGAGCAAGGTGCTGCACCTGGAGGGC
GAGGTGAACAAGATCAAGAGCGCCCTGCTGAGCACCAACAAGGCCGTGGTGAGC
CTGAGCAACGGCGTGAGCGTGCTGACCAGCAAGGTGCTGGACCTGAAGAACTAC
ATCGACAAGCAGCTGCTGCCCATCGTGAACAAGCAGAGCTGCAGCATCCCCAAC
ATCGAGACCGTGATCGAGTTCCAGCAGAAGAACAACAGACTGCTGGAGATCACC
AGAGAGTTCAGCGTGAACGCCGGCGTGACCACCCCCGTGAGCACCTACATGCTG
ACCAACAGCGAGCTGCTGAGCCTGATCAACGACATGCCCATCACCAACGACCAG
AAGAAGCTGATGAGCAACAACGTGCAGATCGTGAGACAGCAGAGCTACAGCATC
ATGAGCATCATCAAGGAGGAGGTGCTGGCCTACGTGGTGCAGCTGCCCCTGTAC
GGCGTGATCGACACCCCCTGCTGGAAGCTGCACACCAGCCCCCTGTGCACCACC
AACACCAAGGAGGGCAGCAACATCTGCCTGACCAGAACCGACAGAGGCTGGTAC
TGCGACAACGCCGGCAGCGTGAGCTTCTTCCCCCAGGCCGAGACCTGCAAGGTG
CAGAGCAACAGAGTGTTCTGCGACACCATGAACAGCCTGACCCTGCCCAGCGAG
GTGAACCTGTGCAACGTGGACATCTTCAACCCCAAGTACGACTGCAAGATCATG
ACCAGCAAGACCGACGTGAGCAGCAGCGTGATCACCAGCCTGGGCGCCATCGTG
AGCTGCTACGGCAAGACCAAGTGCACCGCCAGCAACAAGAACAGAGGCATCATC
AAGACCTTCAGCAACGGCTGCGACTACGTGAGCAACAAGGGCGTGGACACCGTG
AGCGTGGGCAACACCCTGTACTACGTGAACAAGCAGGAGGGCAAGAGCCTGTAC
GTGAAGGGCGAGCCCATCATCAACTTCTACGACCCCCTGGTGTTCCCCAGCGACG
AGTTCGACGCCAGCATCAGCCAGGTGAACGAGAAGATCAACCAGAGCCTGGCCT
TCATCAGAAAGAGCGACGAGCTGCTGCACAACGTGAACGCCGTGAAGAGCACCA
CCAACATCATGATCACCACCATCATCATCGTGATCATCGTGATCCTGCTGAGCCT
GATCGCCGTGGGCCTGCTGCTGTACTGCAAGGCCAGAAGCACCCCCGTGACCCT
GAGCAAGGACCAGCTGAGCGGCATCAACAACATCGCCTTCAGCAACTGA
(RSV F pre-F2.2)
SEQ ID NO: 7
ATGGAGCTGCTGATCCTGAAGGCCAACGCCATCACCACCATCCTGACCGCCGTG
ACCTTCTGCTTCGCCAGCGGCCAGAACATCACCGAGGAGTTCTACCAGAGCACCT
GCAGCGCCGTGAGCAAGGGCTACCTGAGCGCCCTGAGAACCGGCTGGTACACCA
GCGTGATCACCATCGAGCTGAGCAACATCAAGGAGATCAAGTGCAACGGCACCG
ACGCCAAGGTGAAGCTGATCAAGCAGGAGCTGGACAAGTACAAGAACGCCGTG
ACCGAGCTGCAGCTGCTGATGCAGAGCACCCCCGCCACCAACAACAGAGCCAGA
AGAGAGCTGCCCAGATTCATGAACTACACCCTGAACAACGCCAAGAAGACCAAC
GTGACCCTGAGCAAGAAGAGAAAGAGAAGATTCCTGGGCTTCCTGCTGGGCGTG
GGCAGCGCCATCGCCAGCGGCGTGGCCGTGAGCAAGGTGCTGCACCTGGAGGGC
GAGGTGAACAAGATCAAGAGCGCCCTGCTGAGCACCAACAAGGCCGTGGTGAGC
CTGAGCAACGGCGTGAGCGTGCTGACCAGCAAGGTGCTGGACCTGAAGAACTAC
ATCGACAAGCAGCTGCTGCCCATCGTGAACAAGCAGAGCTGCAGCATCCCCAAC
ATCGAGACCGTGATCGAGTTCCAGCAGAAGAACAACAGACTGCTGGAGATCACC
AGAGAGTTCAGCGTGAACGCCGGCGTGACCACCCCCGTGAGCACCTACATGCTG
ACCAACAGCGAGCTGCTGAGCCTGATCAACGACATGCCCATCACCAACGACCAG
AAGAAGCTGATGAGCAACAACGTGCAGATCGTGAGACAGCAGAGCTACAGCATC
ATGAGCATCATCAAGGAGGAGGTGCTGGCCTACGTGGTGCAGCTGCCCCTGTAC
GGCGTGATCGACACCCCCTGCTGGAAGCTGCACACCAGCCCCCTGTGCACCACC
AACACCAAGGAGGGCAGCAACATCTGCCTGACCAGAACCGACAGAGGCTGGTAC
TGCGACAACGCCGGCAGCGTGAGCTTCTTCCCCCAGGCCGAGACCTGCAAGGTG
CAGAGCAACAGAGTGTTCTGCGACACCATGAACAGCCTGACCCTGCCCAGCGAG
GTGAACCTGTGCAACGTGGACATCTTCAACCCCAAGTACGACTGCAAGATCATG
ACCAGCAAGACCGACGTGAGCAGCAGCGTGATCACCAGCCTGGGCGCCATCGTG
AGCTGCTACGGCAAGACCAAGTGCACCGCCAGCAACAAGAACAGAGGCATCATC
AAGACCTTCAGCAACGGCTGCGACTACGTGAGCAACAAGGGCGTGGACACCGTG
AGCGTGGGCAACACCCTGTACTACGTGAACAAGCAGGAGGGCAAGAGCCTGTAC
GTGAAGGGCGAGCCCATCATCAACTTCTACGACCCCCTGGTGTTCCCCAGCAACG
AGTTCGACGCCAGCATCAGCCAGGTGAACGAGAAGATCAACCAGAGCCTGGCCT
TCATCAGAAAGAGCGACGAGCTGCTGCACAACGTGAACGCCGTGAAGAGCACCA
CCAACATCATGATCACCACCATCATCATCGTGATCATCGTGATCCTGCTGAGCCT
GATCGCCGTGGGCCTGCTGCTGTACTGCAAGGCCAGAAGCACCCCCGTGACCCT
GAGCAAGGACCAGCTGAGCGGCATCAACAACATCGCCTTCAGCAACTGA

1. A method of inducing both a protective immune response against respiratory syncytial virus (RSV) infection and a protective immune response against influenza virus infection in a human subject in need thereof, comprising intramuscularly administering to the subject:
(a) an effective amount of a pharmaceutical composition, a vaccine, comprising an adenoviral vector comprising a nucleic acid encoding an RSV F polypeptide that is stabilized in a pre-fusion conformation, wherein the effective amount of the pharmaceutical composition comprises about 1×10¹⁰ to about 1×10¹² viral particles of the adenoviral vector per dose; and
(b) an effective amount of an influenza vaccine, a seasonal influenza vaccine,
wherein (a) and (b) are co-administered.

2. The method of claim 1, wherein the pharmaceutical composition of (a) and the vaccine of (b) are administered at the same time.

3. The method of claim 1, wherein the adenoviral vector is replication-incompetent and has a deletion in at least one of the adenoviral early region 1 (E1 region) and the early region 3 (E3 region).

4. The method of claim 3, wherein the adenoviral vector is a replication-incompetent Ad26 adenoviral vector having a deletion of the E1 region and the E3 region.

5. The method of claim 3, wherein the adenoviral vector is a replication-incompetent Ad35 adenoviral vector having a deletion of the E1 region and the E3 region.

6. The method of claim 1, wherein the recombinant RSV F polypeptide encoded by the adenoviral vector has the amino acid sequence of SEQ ID NO: 4 or SEQ ID NO: 5.

7. The method of claim 1, wherein the nucleic acid encoding the RSV F polypeptide comprises the polynucleotide sequence of SEQ ID NO: 6 or SEQ ID NO: 7.

8. The method of claim 1, wherein the effective amount of the pharmaceutical composition comprises about 1×10¹¹ viral particles of the adenoviral vector per dose.

9. The method of claim 1, wherein the subject is susceptible to the RSV infection.

10. The method of claim 1, wherein the subject is susceptible to the influenza virus infection.

11. The method of claim 1, wherein the protective immune response is characterized by an absent or reduced RSV clinical symptom in the subject upon exposure to RSV.

12. The method of claim 1, wherein the protective immune response is characterized by an absent or reduced influenza virus clinical symptom in the subject upon exposure to influenza virus.

13. The method of claim 1, wherein the protective immune response is characterized by the presence of neutralizing antibodies to RSV and/or protective immunity against RSV, detectible 8 to 35 days after administration of the pharmaceutical composition and the vaccine.

14. The method of claim 1, wherein the protective immune response is characterized by the presence of neutralizing antibodies to influenza virus and/or protective immunity against influenza virus, detectible 8 to 35 days after administration of the pharmaceutical composition and the vaccine.

15. The method of claim 1, wherein the administration does not induce any severe adverse event.

16. A combination comprising:
(a) an effective amount of a pharmaceutical composition, a vaccine, comprising an adenoviral vector comprising a nucleic acid encoding a respiratory syncytial virus (RSV) F polypeptide stabilized in a pre-fusion conformation, wherein the effective amount of the pharmaceutical composition comprises about 1×10¹⁰ to about 1×10¹² viral particles of the adenoviral vector per dose; and
(b) an effective amount of an influenza vaccine, a seasonal influenza vaccine,
for use in intramuscular administration to a human subject in need thereof to induce both a protective immune response against RSV infection and a protective immune response against influenza virus infection in the human subject, wherein (a) and (b) are co-administered.
